(19) United States
(12) Patent Application Publication
(10) Pub. No.: US 2026/0325718 A1
(43) Pub. Date: Oct. 22, 2026

(54) An Aqueous Bioink Solution for Use in Light-Based Bioprinting Applications, and Methods of Using the Aqueous Bioink Solution

(71) Applicants: Cellink Bioprinting AB, Göteborg (SE); Allegro 3D Inc., San Diego, CA (US)

(72) Inventor: Wei Zhu, San Diego, CA (US)

(21) Appl. No.: 19/122,069

(22) PCT Filed: Oct. 20, 2023
(86) PCT No.: PCT/EP2023/079273

§ 371 Date: Apr. 17, 2025

Related U.S. Application Data
(60) Provisional application No. 63/417,805, filed on Oct. 20, 2022.

(51) Int. Cl. A61L 27/52; A61L 27/16; A61L 27/38; B33Y 70/00

(52) U.S. Cl. CPC A61L 27/52; A61L 27/16; A61L 27/38; B33Y 70/00

(57) ABSTRACT

The disclosure relates to an aqueous bioink solution for use in light-based bioprinting applications, comprising: (a) 0.5-95 wt % water-soluble prepolymer; (b) 0.001-5 wt % biocompatible metal acylphosphinate photoinitiator; (c) 0.001-10 wt % biocompatible light absorber; and (d) 5 to 99.5 wt % solvent. The disclosure further relates to a method of using the aqueous bioink solution including living cells and a method of using the aqueous bioink solution without living cells (acellularly).

### TECHNICAL FIELD

The present disclosure relates to an aqueous bioink solution for use in light-based bioprinting applications, a method of using the aqueous bioink solution including live cells and a method of using the aqueous bioink solution without live cells (acellularly). More specifically, the disclosure relates to an aqueous bioink solution for use in light-based bioprinting applications, and methods of using the aqueous bioink solution as defined in the introductory parts of the independent claims.

### BACKGROUND ART

In recent years, 3D bioprinting technologies have become the forefront in innovative tools to create customizable scaffolds or tissues with varying degrees of complexity for applications in tissue engineering, regenerative medicine, microfluidics, drug discovery, etc. There are various modalities of 3D bioprinting platforms, such as inkjet bioprinting, extrusion bioprinting, digital light processing (DLP) bioprinting, laser assisted bioprinting [1]. Among them, DLP bioprinting stands out for its superior printing speed, resolution, scalability, and throughput in creating highly complex 3D tissues, biomimetic scaffolds as well as microfluidic chips.

DLP bioprinting employs a digital micromirror device (DMD), composed of millions of micromirrors, to modulate the light patterns projected onto the photopolymer solution. The exposed area of the photopolymer solution will be photo-crosslinked. By coordinating the change of the projected light patterns and the movement of a motorized stage that carries the sample being printed, a 3D structure can be printed either layer-by-layer or continuously [2-6]. The design of the projected light patterns can be derived from computer aided design (CAD) models as well as medical imaging data such as magnetic resonance imaging (MRI) and computerized tomography (CT) scans, enabling the fabrication of highly complex biomedical devices (e.g., microfluidic chips) and patient-specific tissue models or implants.

The quality or fidelity of the printed structure is directly associated with the printing resolution in both lateral and axial directions. While lateral resolution can be better controlled by the projection optics to reach microscale or even submicron scale (2 μm lateral resolution has been commercially achieved in DLP printing), the axial resolution is dictated by the projected light penetration depth in the photopolymer solution which is typically 10 to 100-fold worse than the axial resolution (e.g., typical axial resolution of DLP printing=hundreds of microns to millimeters) [7]. To improve the axial resolution in light-based bioprinting (including both DLP bioprinting and laser assisted bioprinting), light absorbers (e.g., tartrazine, quinoline yellow) have been added to the prepolymer solution to reduce the light penetration depth [6].

Another key component of the prepolymer solution for light-based bioprinting (including DLP bioprinting and laser assisted bioprinting) is the photoinitiator. Because the prepolymer solution for bioprinting is typically aqueous to allow incorporation of live cells during or post the bioprinting process, the photoinitiator must be water soluble. More importantly, to bioprint functional tissues and scaffolds for in vitro and in vivo biomedical applications (e.g., tissue engineering, drug testing, transplantation, cell and tissue therapies, etc.), the photoinitiator must be biocompatible with minimal cytotoxicity and minimal adverse side effects to human health.

Lithium acylphosphinate (LAP), a type of lithium salt, has been used together with light absorbers (i.e., tartrazine, quinoline yellow) in the aqueous prepolymer solution for fabricating complex 3D hydrogels [6,8,9]. However, lithium salts are known to cause kidney injury and adverse effects to the neurological and renal systems of the human body [10,11]. There are also studies that found LAP can potentially damage the cell membrane, resulting in compromised cell viability after light-based bioprinting [10,11]. Lithium ions are known to accumulate in the kidney collecting duct cells leading to kidney injury and disease, which is a common side effect of lithium treatment for psychiatric disorders [10,12,13]. The cause of such lithium cytotoxicity is that the influx sodium channels on the cell membranes have higher affinity for lithium ions than sodium ions, but the sodium efflux ATPase pumps have lower affinity for lithium than sodium, which leads to excess accumulation of lithium within the cells [10, 12, 13].

Thus, there is a need in the art to provide alternative and/or improved bioink solutions, meeting the high demands on resolution and biocompatibility within the art of light-based 3D printing. Further, there is a demand for alternative and/or improved bioink solutions having a low cytotoxicity, thereby allowing the use of live cellular material in the bioink solution. Still further, there is a need for alternative and/or improved photoinitiators as well as a need for alternative, and/or improved light absorbers for the fabrication of complex 3D structures.

### SUMMARY

It is an object of the present disclosure to mitigate, alleviate or eliminate one or more of the above-identified deficiencies and disadvantages in the prior art and solve at least the above-mentioned problem(s) and need(s).

An unexpected solution to eliminate the cytotoxicity associated with lithium is to use metal ions that naturally exist in human body fluid to replace lithium in the metal-acylphosphinate (Metal-AP) photoinitiators, thereby obtaining an improved biocompatibility. Such physiologically relevant metal ions include, but are not limited to, sodium, magnesium, calcium, and potassium. While sodium and magnesium acylphosphinates have been synthesized in literature [9], to the inventor's knowledge, there have been no published studies that combine them with light absorber in a prepolymer solution to bioprint high-fidelity complex 3D acellular scaffolds or cell-laden tissue constructs using light based bioprinting platforms for tissue engineering and regenerative medicine applications. Also, there are no commercial products available for any other metal-acylphosphinates than those including lithium.

According to a first aspect there is provided an aqueous bioink solution for use in light-based bioprinting applications, comprising: 0.5-95 w/v (%) water-soluble photocrosslinkable prepolymer; 0.001-5 w/v (%) biocompatible metal acylphosphinate photoinitiator; 0.001-10 w/v (%) biocompatible light absorber; and 5 to 99.5 w/v (%) solvent.

According to a second aspect there is provided a method of using the aqueous bioink solution according to the first aspect on light-based bioprinting platforms for the creation of cell-laden tissues or hydrogel constructs or scaffolds, such as for cell encapsulation, seeding, in vitro tissue engineering, disease modelling or drug discovery, wherein cells are mixed with the aqueous bioink solution to the desired concentration and then loaded to the bioprinting platform to create 3D tissues, hydrogel constructs or scaffolds containing cells, and thereafter using the 3D tissues, hydrogel constructs or scaffolds containing cells for cell encapsulation, seeding, in vitro tissue engineering, disease modelling or drug discovery.

According to a third aspect there is provided a method of using the aqueous bioink solution according to the first aspect on light-based bioprinting platforms to create acellular tissues or hydrogel constructs or scaffolds, such as for in vivo implantation, in vitro cell seeding and therapeutic applications, wherein the aqueous bioink solution with desired composition is loaded directly on the light-based bioprinting platforms to create acellular 3D tissues, hydrogel constructs or scaffolds and thereafter using the 3D tissues, hydrogel constructs or scaffolds for in vivo implantation, in vitro cell seeding or therapeutic applications.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### Definitions

The shortage “AP” refers to acylphosphinate(s).

By a “biocompatible” material is meant that the material is not harmful or toxic to living tissue. The biocompatibility of a material, such as a bioink or ingredients of a bioink, may depend on e.g., the cell type, other components of the bioink and printing conditions.

By “tissues, hydrogel constructs or scaffolds” is, in the context of this disclosure, meant any 3D-bioprinted product or structure, with or without cells, produced by using the aqueous bioink solution of the present disclosure.

Concentration values of the ingredients of the aqueous bioink solution of this disclosure are given in weight/volume of the final bioink solution, also abbreviated as w/v (%).

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 shows Live/dead viability results of C2C12 cells bioprinted in Na-AP (left) and LAP (right) based bioinks. Cells in Na-AP based bioink demonstrated healthier morphology than those in LAP based bioink at Day 1 post bioprinting.

FIG. 2 shows printing results comparing Na-AP vs LAP based bioinks.

FIG. 3 discloses devices and 3D structures printed with bioinks composed of two types of PEGDA, Na-AP and light absorber using a DLP bioprinter. (a-b) Microfluidic chips with horizontal channels. The hollow horizonal channels in the devices were perfused with red and blue dyes to demonstrate the high printing fidelity. (c) A free-standing lattice structure. (d) A free-standing mesh stent.

### DETAILED DESCRIPTION

The present description provides an improved aqueous bioink solution for use in light-based bioprinting applications, a method of using the aqueous bioink solution including live cells and a method of using the aqueous bioink solution without live cells (acellularly). According to an example embodiment there is provided an aqueous bioink solution for use in light-based bioprinting applications, comprising: 0.5-95 w/v (%) water-soluble photocrosslinkable prepolymer; 0.001-5 w/v (%) biocompatible metal acylphosphinate photoinitiator; 0.001-10 w/v (%) biocompatible light absorber; and 5 to 99.5 w/v (%) solvent.

Thus, the present disclosure relates to biocompatible aqueous bioink solutions with at least a water soluble photocrosslinkable prepolymer, a biocompatible metal acylphosphinate photoinitiator and a biocompatible light absorber.

Hereby, an aqueous and biocompatible bioink solution for light-based bioprinting applications is provided, which solution exhibit promising results in terms of bioprinting results, cell survival/health, cell differentiation and maturation.

According to an example, the biocompatible metal acylphosphinate photoinitiator is chosen from the group comprising Na-AP, Mg-AP, Ca-AP and K-AP.

These metal acyl phosphinate photoinitiators are all promising and previously unknown candidates as photoinitiators.

According to an example, the biocompatible metal acylphosphinate photoinitiator is Na-AP. Especially, Na-AP has shown promising results in relation to the previously known alternative LAP.

According to an example, the biocompatible light absorber is chosen from the group comprising quinoline yellow, ponceau 4r, sunset yellow, yellow food dyes, micro/nanoparticles, of different sizes and types, riboflavin, phenol red, curcumin, saffron, turmeric, beta carotene, carbon black and tartrazine.

These light absorbers are all promising candidates as light absorbers.

In one example, the biocompatible light absorber is quinoline yellow. Hereby, since potential toxic effects as for other regularly used light absorbers, such as e.g., tartrazine [14], does not appear to be known for quinoline yellow, an improved biocompatibility is obtained.

Moreover, it has been identified that quinoline yellow may exhibit a higher absorbance at 405 nm per gram than other light absorbers, such as tartrazine. Thus, quinoline yellow may be used at a lower concentration than e.g., tartrazine, resulting in less material to be removed from the construct for better imaging, and/or less negative effects (if any).

Further, in one example the biocompatible metal acylphosphinate photoinitiator is Na-AP and the biocompatible light absorber is quinoline yellow. Hereby, a bioink having an improved toxicity profile, while maintaining or improving the bioprinting properties in light-based bioprinting applications, is provided. Thus, by exchanging the metal ion lithium with the very prevalent sodium ion, in combination with using quinoline yellow as the light absorber, an improved biocompatibility behaviour is obtained.

According to an example, Na-AP is used in a concentration of 0.001-5.0% wt, or 0.01-2% wt, or 0.1-1% wt, or 0.5-1.0% wt, and quinoline yellow is used in a concentration of 0.001-10% wt, or 0.01-1% wt, or 0.05-0.2% wt, respectively. A bioink wherein Na-AP is used in a concentration of 0.5-1% wt and quinoline yellow is used in a concentration of 0.05-0.2% wt, respectively, has been identified as one example.

According to an example, the water-soluble prepolymer is chosen from monomers or oligomers with photocrosslinkable moieties such as vinyl, acrylic, arylamide, acrylate, thiol-ene and epoxide.

Thus, by using water-soluble prepolymer including photocrosslinkable moieties, a polymerizing effect is obtained.

According to an example, the water-soluble prepolymer is chosen from poly(ethylene glycol) diacrylate, gelatin methacrylate, methacrylated hyaluronic acid, methacrylated alginate, methacrylated silk, and mixtures of two or more of PEGDA, GelMA, HAMA, AlgMA and Silk-MA.

According to an example, PEGDA is chosen from the group comprising 700, 1K, 3.4K, 6K, 10K and 20K MW. Other PEGDA prepolymer molecular weights may also be used.

Hereby, a suitable water-soluble prepolymer is provided.

According to an example, the solvent is chosen from the group comprising water, phosphate-buffered saline and cell culture medium. Other solvents may also be used, depending on application and context. Typically, cell culture media may comprise ingredients such as carbohydrates, amino acids, vitamins, minerals, and a pH buffer system. Other ingredients may also be included, e.g., depending on application and cell type.

Hereby, a suitable solvent is provided.

TABLE 1
lists representative ink formulations for the disclosed
biocompatible aqueous bioink solutions.

| Key | Examples of key components |
|---|---|
| components | and typical concentration ranges (w/v (%)) |
| Water soluble | Poly(ethylene glycol) diacrylate (PEGDA)- |
| prepolymer | 700, 1K, 3.4K, 6K, 10K, 20K MW |
| | 5%-95% |
| | Gelatin methacrylate (GelMA) |
| | 0.5%-25% |
| | Methacrylated Hyaluronic Acid (HAMA) |
| | 0.5%-25% |
| | Methacrylated Alginate (AlgMA) |
| | 0.5%-25% |
| | Methacylated silk (Silk-MA) |
| | 0.5%-25% |
| | Mixtures of the above, e.g., PEGDA |
| | (MW 700) + PEGDA (MW 6K), |
| | PEGDA + GelMA, GelMA + HAMA, etc. |
| Biocompatible | Na-AP, Mg-AP, Ca-AP, K-AP |
| metal | 0.001%-5% |
| acylphosphinate | |
| photoinitiator | |
| Biocompatible | Quinoline yellow, ponceau 4R, sunset |
| light absorber | yellow, yellow food dyes, micro/ |
| | nanoparticles, riboflavin, phenol red, curcumin, |
| | saffron, turmeric, beta carotene, carbon black, |
| | tartrazine etc. 0.001%-10% |
| Solvent | Water, PBS, cell culture medium |
| | 5%-99.5% |

According to an example, the solution further comprises living cells.

Thus, the aqueous bioink solutions can be used in applications requiring living cells, which can be added prior to or after bioprinting depending on application.

According to an example, the living cells are chosen from the group comprising cells or cell lines of human and/or animal origin, being primary cells, immortalized and iPSC- or ESC-derived, such as keratinocytes, melanocytes, fibroblasts, sebocytes, dendritic cells, macrophages, stem cells, induced pluripotent stem cells, adipocytes, glandular cells or follicle cells, as well as myoblast cells, hepatic cells, human primary renal proximal tubule epithelial cells, collecting duct cells or adipose-derived mesenchymal stem cells. Any other cells and/or cell types may also be used. The cells may for example be related to dermal, hepatic, cardiac, kidney, lung, muscle, cartilage or neural tissue. Any other tissue types may also be used.

Thus, any suitable cells and/or cells of interest may be included in the bioink solution of the present disclosure.

According to an example, the aqueous bioink solution may comprise one or more additional ingredients, such as cryoprotectants, DMEM media, serum, proteins, lipids, nucleic acids, carbohydrates, hormones, glycosaminoglycans, collagens, methacrylate collagen, gelatin, cellulose, nanofibrillar cellulose, alginate, chitosan, acacia gum, tara gum, glucomannan, pectin, locust bean gum, guar gum, carrageenan or tragacanth, elastin, proteoglycans, aggrecans, isolated laminins, glycol-aminoglycans, such as hyaluronic acid and heparin, growth factors, thickeners, such as a polysaccharide-based substance, such as nanocellulose, glucomannan, xanthan gum, gellan gum, diutan gum, welan gum or pullalun gum, or a protein-based substance, such as collagen or gelatin, additional biopolymers, extracellular matrix components, decellularized matrix components, factor A or any other ingredient that is useful for the purposes of the present disclosure.

### Providing and Mixing of the Bioink Solution

A typical bioink for the present disclosure is produced by mixing the selected components from the above table. Some of the components (e.g., prepolymers, light absorbers, solvents and any additional ingredients) would typically be commercially available and/or obtainable by methods and processes known for a skilled person. For details on obtaining metal acylphosphinate photoinitiators, see e.g., [9], which is hereby incorporated as a reference.

### Light-Based Bioprinting

As discussed in the background section, light-based bioprinting refers to methods utilizing light and/or a light pattern to photo-crosslink 3D printed layers and structures, including digital light processing (DLP) bioprinting, laser assisted bioprinting, stereolithography (SLA) bioprinting and two photon polymerization (TPP) bioprinting. For example, in this context it is referred to US2017/0087766, US2016/0298087, WO2020/210681 and WO2021/087281, which disclosures are incorporated herein as references.

### Use of the Bioink

The disclosed biocompatible aqueous bioink solutions can be used on light-based bioprinting platforms to create hydrogel constructs for cell encapsulation or seeding for in vitro tissue engineering, disease modelling, drug discovery, etc.

The above disclosed biocompatible aqueous bioink solutions can also be used on light-based bioprinting platforms to create cell-laden tissues or acellular hydrogel constructs for in vivo implantation and therapeutic applications.

For cell encapsulation, cells will be mixed with the bioink solution to the desired concentrations and then loaded to the bioprinting platform to create 3D tissues. For acellular scaffolds, bioink solution with desired compositions can be loaded directly on the light-based bioprinting platforms to create 3D scaffolds, which can be subsequently used for in vivo implantation or in vitro cell seeding.

Further, the disclosed biocompatible aqueous bioink solutions can be used for fabrication of various 3D tissue models, such as cardiac tissues, kidney tissues, lung tissues, nerve tissues, muscle tissues, cartilage tissues, skin tissues, hepatic tissues etc. Such tissue products can feature biomimetic 3D geometries as well as cellular compositions mimicking native tissues and in vivo environment, which cannot be achieved by conventional 2D cell cultures.

Thus, according to an example embodiment there is provided a method of using the aqueous bioink solution according to the first aspect on light-based bioprinting platforms for the creation of cell-laden tissues, hydrogel constructs or scaffolds, such as for cell encapsulation, seeding, in vitro tissue engineering, disease modelling or drug discovery, wherein cells are mixed with the aqueous bioink solution to the desired concentration and then loaded to the bioprinting platform to create 3D tissues, hydrogel constructs or scaffolds containing cells, and thereafter using the 3D tissues, hydrogel constructs or scaffolds containing cells for cell encapsulation, seeding, in vitro tissue engineering, disease modelling or drug discovery.

Hereby, a method offering use and subsequent production of cell-including 3D-tissues, constructs or scaffolds is provided.

According to an example embodiment there is provided a method of using the aqueous bioink solution according to the first aspect on light-based bioprinting platforms to create acellular tissues, hydrogel constructs or scaffolds, such as for in vivo implantation, in vitro cell seeding and therapeutic applications, wherein the aqueous bioink solution with desired composition is loaded directly on the light-based bioprinting platforms to create acellular 3D tissues, hydrogel constructs or scaffolds and thereafter using the 3D tissues, hydrogel constructs or scaffolds for in vivo implantation, in vitro cell seeding or therapeutic applications.

Hereby, a method offering use and subsequent production of acellular 3D-tissues, constructs or scaffolds is provided.

The present disclosure will now be described with reference to the accompanying examples, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

### EXAMPLES

### Experimental Results

1. Cytotoxicity of Na-AP/Mg-AP/Ca-AP/K-AP vs. LAP

A first novel aspect of the present disclosure is the use of Na-AP, Mg-AP, Ca-AP, and K-AP as photoinitiator in the disclosed bioinks, which are more biocompatible and physiologically relevant photoinitiators than LAP. Na, Mg, Ca, and K are ions that naturally exist in human body fluid and are essential nutrients for human body. They are also essential components in cell culture medium. Therefore, using Na-AP, Mg-AP, Ca-AP, and K-AP as a photoinitiator supports better cell viability, proliferation, and differentiation than LAP.

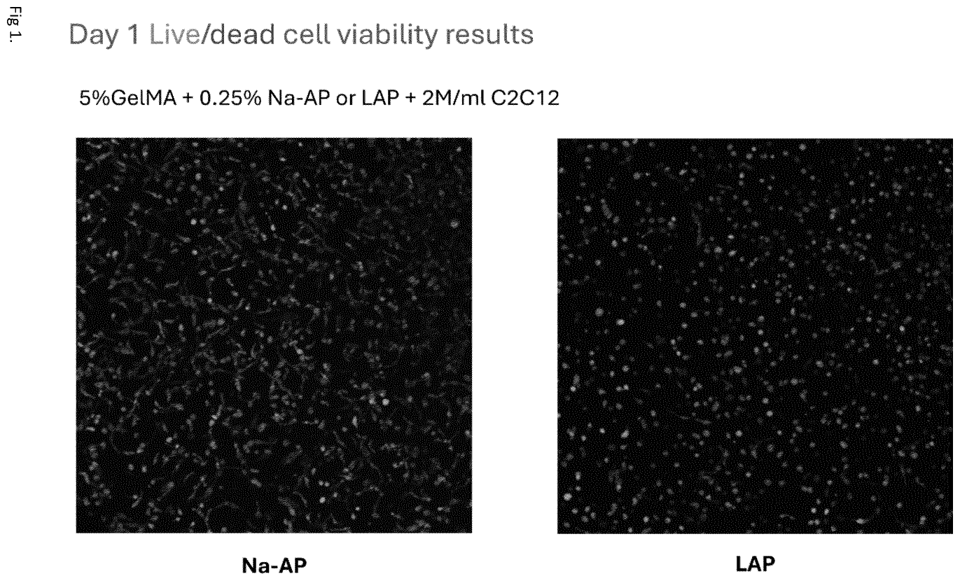

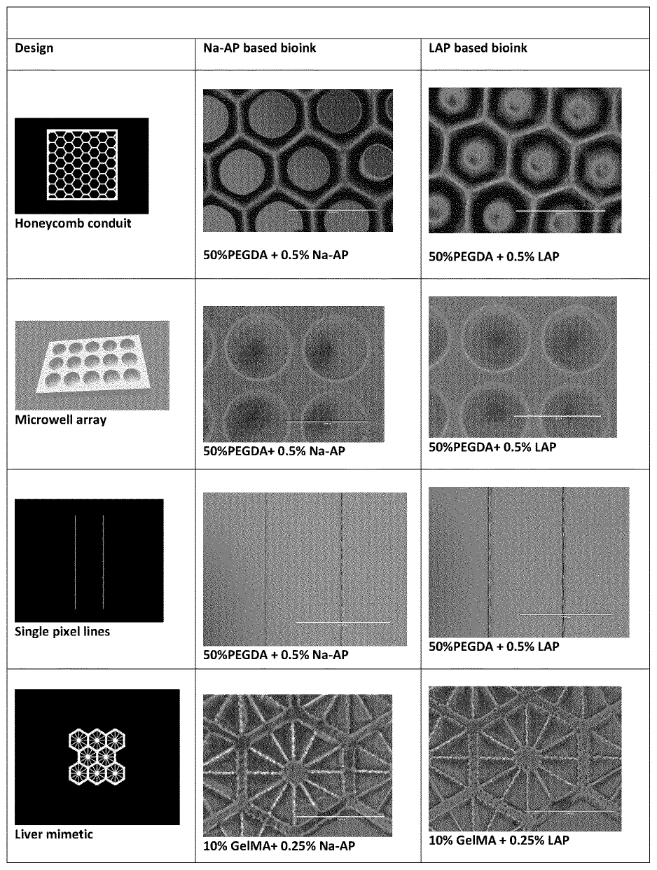

The inventor used Na-AP as an example to compare its cytotoxicity with LAP. The inventor formulated two bioinks to bioprint tissues encapsulated with myoblast cells (i.e., C2C12): (1) 5% GelMA+0.25% Na-AP+2 million/ml C2C12 cells (a type of myoblast cell); (2) 5% GelMA+0.25% LAP+2 million/ml C2C12 cells. A DLP bioprinter (BIONOVA X, from CELLINK) was used to print tissue constructs with these two cell-laden bioinks and compared cell viability on Day 1 post bioprinting. A commercial cytotoxicity kit (LIVE/DEAD™ Viability/Cytotoxicity Kit for mammalian cells, Catalog #L3224, ThermoFisher Scientific) was used to evaluate the cell viability. Staining and imaging were performed according to manufacturer's recommendations. As shown in FIG. 1, the cells encapsulated in the Na-AP based bioink demonstrated healthier cell morphology (cells more spread out) than the cells in the LAP based bioink (cells balled up).

The inventors have also planned using other cells to further compare and systematically investigate the cytotoxicity of LAP vs. Na-AP/Mg-AP/Ca-AP/K-AP. Some representative cell types include but are not limited to, human primary renal proximal tubule epithelial cells and collecting duct cells, as lithium is known to target the renal system and kidney collecting duct cells. The cells will be exposed to following experimental conditions to evaluate their viability, morphology, proliferation, and metabolism:
1) Cells will be incubated directly in bioinks made with different photoinitiators (i.e., LAP vs Na-AP vs Mg-AP vs Ca-AP vs. K-AP) for a certain time (e.g., 6, 12, 24 hours) before assaying. Except for the photoinitiator, all the other components of the bioinks (i.e., prepolymer, light absorber, solvent, etc.) will be controlled to be the same;
2) Cells will be mixed in bioinks made with different photoinitiators (i.e., LAP vs Na-AP vs Mg-AP vs Ca-AP vs. K-AP) and immediately printed into tissues with light-based bioprinting (e.g., DLP bioprinting). Except for the photoinitiator, all the other components of the bioinks (i.e., prepolymer, light absorber, solvent, etc.) will be controlled to be the same. The printed tissues will be assayed after a certain time of in vitro culturing (e.g. 1, 4, 7 days after bioprinting).

Commercial cell assay kits that can be used to evaluate the viability, morphology, proliferation, and metabolism include but are not limited to LIVE/DEAD™ Viability/Cytotoxicity Kit, Alamar Blue assay, CyQuant Direct cell assay, Neutral Red Uptake cytotoxicity assay, MTT assay, Cell Painting assay, etc.

It is expected that cells exposed to Na-AP/Mg-AP/Ca-AP/K-AP based bioinks will present significantly better results in the viability, morphology, proliferation, or metabolism assays as compared to those exposed to the LAP-based bioinks.

2. Impact on Cell Differentiation: Na-AP/Mg-AP/Ca-AP/K-AP Vs. LAP

The inventor will also evaluate the impact of different photoinitiators on cell differentiation. The inventor will use Adipose-Derived Mesenchymal Stem Cells (ADSCs, ATCC PCS-500-011) as the representative cell type for this study. Cells will be mixed in bioinks made with different photoinitiators (i.e., LAP vs Na-AP vs Mg-AP vs Ca-AP vs. K-AP) and immediately printed into tissues with light-based bioprinting (e.g., DLP bioprinting). Except for the photoinitiator, all the other components of the bioinks (i.e., prepolymer, light absorber, solvent, etc.) will be controlled to be the same. Cells in the printed tissues will be induced to differentiate into adipocytes using the Adipocyte Differentiation Toolkit (ATCC PCS-500-050). Based on the inventor's current understanding, better differentiation and maturation results (e.g., higher expression of adipogenesis markers and genes) from ADSCs printed in bioinks made with Na-AP/Mg-AP/Ca-AP/K-AP are expected, as compared to those in LAP-based bioinks.

3. Photoinitiator Efficiency in Light-Based Bioprinting: Na-AP/Mg-AP/Ca-AP/K-AP Vs. LAP

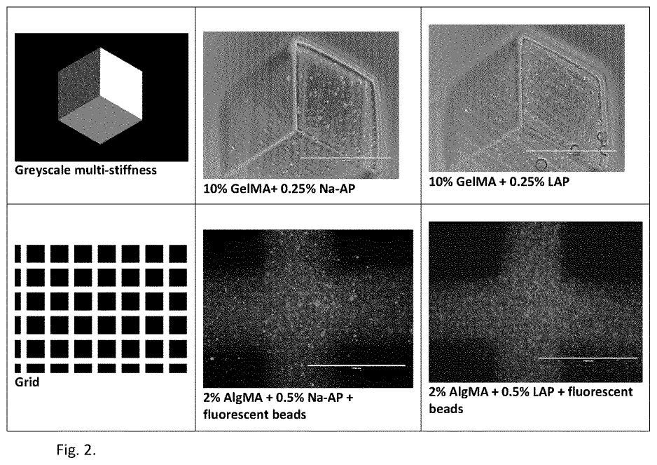

To compare their efficiency in light-based bioprinting, the inventor formulated bioinks with different photoinitiators (e.g., Na-AP vs. LAP) while keeping the other components the same. The inventor then used these bioinks to print the same design with the same light-based bioprinting platform and parameters. It was found that Na-AP and LAP have comparable efficiency and performance in light-based bioprinting, as shown in FIG. 2.

4. DLP Bioprinting with Na-AP/Mg-AP/Ca-AP/K-AP Based Bioinks with Light Absorber Vs. No Light Absorber

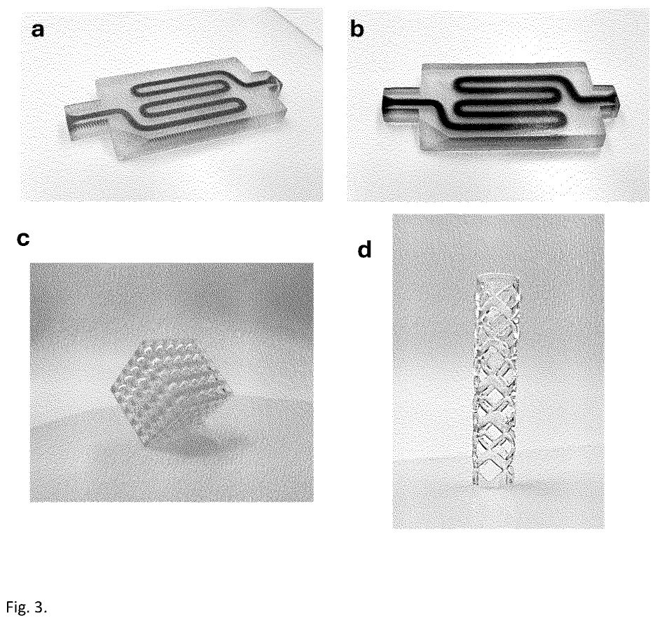

Although Na-AP/Mg-AP/Ca-AP/K-AP based bioinks without light absorber can be used for light-based printing, there is no control over the light penetration depth in axial direction, leading to very poor axial resolution (i.e., hundreds of microns to a few millimeters). The low axial resolution limits the use of such bioinks without light absorber to only printing 2.5D structures with no or minimal design variation in the axial direction, such as the printing results shown in FIG. 2. Without a higher axial resolution, complex 3D designs such as overhanging structures and horizontal hollow channels cannot be printed using light-based printing, especially DLP-based bioprinting. Nevertheless, such complex 3D designs are essential to engineer in vitro functional human tissues (e.g., vasculature, nerve, etc.), microfluidic devices, organ-on-a-chip systems, etc. To address these challenges, a biocompatible light absorber can be added to the bioink to reduce light penetration in the bioink solution and improve axial resolution. Such biocompatible light absorbers include but are not limited to Quinoline yellow, ponceau 4R, sunset yellow, yellow food dyes, micro/nanoparticles, riboflavin, phenol red, curcumin, saffron, turmeric, beta carotene, carbon black, tartrazine etc. To the inventor's knowledge, there is currently no published work using Na-AP/Mg-AP/Ca-AP/K-AP based bioinks with biocompatible light absorbers. This is another novel aspect of the present disclosure. The inventor has demonstrated the printing of complex 3D structures using bioinks with both Na-AP and a light absorber as shown in FIG. 3.

The person skilled in the art realizes that the present disclosure is not limited to the preferred embodiments and examples described above. The person skilled in the art further realizes that modifications and variations are possible within the scope of the appended claims.

Additionally, variations to the disclosed embodiments can be understood and effected by the skilled person in practicing the claimed disclosure, from a study of the drawings, the examples the disclosure, and the appended claims.

### REFERENCES

[1] W. Zhu, X. Ma, M. Gou, D. Mei, K. Zhang, S. Chen, 3D printing of functional biomaterials for tissue engineering, Curr Opin Biotechnol. 40 (2016) 103-112. https://doi.org/10.1016/j.copbio.2016.03.014.
[2] W. Zhu, K. R. Tringale, S. A. Woller, S. You, S. Johnson, H. Shen, J. Schimelman, M. Whitney, J. Steinauer, W. Xu, T. L. Yaksh, Q. T. Nguyen, S. Chen, Rapid continuous 3D printing of customizable peripheral nerve guidance conduits, Materials Today. (2018). https://doi.org/10.1016/j.mattod.2018.04.001.
[3] K. Kim, W. Zhu, X. Qu, C. Aaronson, W. R. McCall, S. Chen, D. J. Sirbuly, 3D optical printing of piezoelectric nanoparticle-polymer composite materials, ACS Nano. 8 (2014) 9799-9806. https://doi.org/10.1021/nn503268f.
[4] a P. Zhang, X. Qu, P. Soman, K. C. Hribar, J. W. Lee, S. Chen, S. He, Rapid fabrication of complex 3D extracellular microenvironments by dynamic optical projection stereolithography, Advanced Materials. 24 (2012) 4266-4270. https://doi.org/10.1002/adma.201202024.
[5] H. H. Hwang, S. You, X. Ma, L. Kwe, G. Victorine, N. Lawrence, X. Wan, H. Shen, W. Zhu, S. Chen, High throughput direct 3D bioprinting in multiwell plates, Biofabrication. 13 (2021) 025007. https://doi.org/10.1088/1758-5090/ab89ca.
[6] B. Grigoryan, S. J. Paulsen, D. C. Corbett, D. W. Sazer, C. L. Fortin, A. J. Zaita, P. T. Greenfield, N.J. Calafat, J. P. Gounley, A. H. Ta, F. Johansson, A. Randles, J. E. Rosenkrantz, J. D. Louis-Rosenberg, P. A. Galie, K. R. Stevens, J. S. Miller, Multivascular networks and functional intravascular topologies within biocompatible hydrogels, Science (1979). 364 (2019). https://doi.org/10.1126/science.aav9750.
[7] M. Shusteff, A. E. M. Browar, B. E. Kelly, J. Henriksson, T. H. Weisgraber, R. M. Panas, N. X. Fang, C. M. Spadaccini, One-step volumetric additive manufacturing of complex polymer structures, Sci Adv. 3 (2017) eaao5496. https://doi.org/10.1126/sciadv.aao5496.
[8] J. Miller, A. Ta, B. Grigoryan, Hypothermic 3D bioprinting of living tissues supported by perfusable vasculature, U.S. Pat. No. 10,844,350 B2, 2020 and corresponding published application US2018/0002658.
[9] H. Wang, B. Zhang, J. Zhang, X. He, F. Liu, J. Cui, Z. Lu, G. Hu, J. Yang, Z. Zhou, R. Wang, X. Hou, L. Ma, P. Ren, Q. Ge, P. Li, W. Huang, General one-pot method for preparing highly water-soluble and biocompatible photoinitiators for digital light processing-based 3d printing of hydrogels, ACS Appl Mater Interfaces. 13 (2021). https://doi.org/10.1021/acsami.1c15636.
[10] A. K. Nguyen, P. L. Goering, R. K. Elespuru, S. S. Das, R. J. Narayan, The photoinitiator lithium phenyl (2,4,6-Trimethylbenzoyl) phosphinate with exposure to 405 nm light is cytotoxic to mammalian cells but not mutagenic in bacterial reverse mutation assays, Polymers (Basel). 12 (2020) 1-13. https://doi.org/10.3390/polym12071489.
[11] A. K. Nguyen, P. L. Goering, V. Reipa, R. J. Narayan, Toxicity and photosensitizing assessment of gelatin methacryloyl-based hydrogels photoinitiated with lithium phenyl-2,4,6-trimethylbenzoylphosphinate in human primary renal proximal tubule epithelial cells, Biointerphases. 14 (2019) 021007. https://doi.org/10.1116/1.5095886.
[12] B. M. Christensen, A. M. Zuber, J. Loffing, J. C. Stehle, P. M. T. Deen, B. C. Rossier, E. Hummler, αENaC-mediated lithium absorption promotes nephrogenic diabetes insipidus, Journal of the American Society of Nephrology. 22 (2011). https://doi.org/10.1681/ASN.2010070734.
[13] J. Davis, M. Desmond, M. Berk, Lithium and nephrotoxicity: Unravelling the complex pathophysiological threads of the lightest metal, Nephrology. 23 (2018). https://doi.org/10.1111/nep.13263.
[14] B. M. Soares et al., Effects on DNA Repair in Human Lymphocytes Exposed to the Food Dye Tartrazine Yellow, Anticancer Research March 2015, 35(3), 1465-1474.

1. An aqueous bioink solution for use in light-based bioprinting applications, comprising: (a) 0.5-95 w/v (%) water-soluble photocrosslinkable prepolymer; (b) 0.001-5 w/v (%) biocompatible metal acylphosphinate photoinitiator chosen from the group comprising Na-AP, Mg-AP, Ca-AP and K-AP; (c) 0.001-10 w/v (%) biocompatible light absorber; and (d) 5-99.5 w/v (%) solvent.

2. The aqueous bioink solution according to claim 1, wherein the biocompatible metal acylphosphinate photoinitiator is Na-AP.

3. The aqueous bioink solution according to claim 1, wherein the biocompatible light absorber is chosen from the group comprising quinoline yellow, ponceau 4R, sunset yellow, yellow food dyes, micro/nanoparticles, riboflavin, phenol red, curcumin, saffron, turmeric, beta carotene, carbon black and tartrazine.

4. The aqueous bioink solution according to claim 1, wherein the biocompatible light absorber is quinoline yellow.

5. The aqueous bioink solution according to claim 1, wherein the biocompatible metal acylphosphinate photoinitiator is Na-AP and the biocompatible light absorber is quinoline yellow.

6. The aqueous bioink solution according to claim 5, wherein Na-AP is used at a concentration of 0.5-1% wt and/or quinoline yellow is used at a concentration of 0.05-0.2% wt.

7. The aqueous bioink solution according to claim 1, wherein the water-soluble prepolymer is chosen from monomers or oligomers with photocrosslinkable moieties such as vinyl, acrylic, arylamide, acrylate, thiol-ene and epoxide.

8. The aqueous bioink solution according to claim 1, wherein the water-soluble prepolymer is chosen from poly(ethyleneglycol) diacrylate (PEGDA) (5-95 w/v (%)), gelatin methacrylate (GELMA) (0.5-25 w/v (%)), methacrylated hyaluronic acid (HAMA) (0.5-25 w/v (%)), methacrylated alginate (AlgMA) (0.5-25 w/v (%)), methacrylated silk (Silk-MA) (0.5-25 w/v (%)), and mixtures of two or more of PEGDA, GELMA, HAMA, AlgMA and Silk-MA.

9. The aqueous bioink solution according to claim 8, wherein PEGDA is chosen from the group comprising 700, 1 K, 3.4 K, 6 K, 10 K and 20 K MW.

10. The aqueous bioink solution according to claim 1, wherein the solvent is chosen from the group comprising water, phosphate-buffered saline (PBS) and cell culture medium.

11. The aqueous bioink solution according to claim 1, wherein the solution further comprises living cells.

12. The aqueous bioink solution according to claim 11, wherein the living cells are chosen from the group comprising cells or cell lines of human and/or animal origin, being primary cells, immortalized and iPSC- or ESC-derived, such as keratinocytes, melanocytes, fibroblasts, sebocytes, dendritic cells, macrophages, stem cells, induced pluripotent stem cells, adipocytes, glandular cells or follicle cells, as well as myoblast cells, hepatic cells, human primary renal proximal tubule epithelial cells, collecting duct cells or adipose-derived mesenchymal stem cells; and cells related to dermal, hepatic, cardiac, kidney, lung, muscle, cartilage or neural tissue.

13. A method of using the aqueous bioink solution according to claim 1 on light-based bioprinting platforms for the creation of cell-laden tissues, hydrogel constructs or scaffolds, such as for cell encapsulation, seeding, in vitro tissue engineering, disease modelling or drug discovery, wherein cells are (i) mixed with said aqueous bioink solution to the desired concentration and then (ii) loaded to the bioprinting platform to (iii) create 3D tissues, hydrogel constructs or scaffolds containing cells, and thereafter (iv) using the 3D tissues, hydrogel constructs or scaffolds containing cells for cell encapsulation, seeding, in vitro tissue engineering, disease modelling or drug discovery.

14. A method of using the aqueous bioink solution according to claim 1 on light-based bioprinting platforms to create acellular tissues, hydrogel constructs or scaffolds, such as for in vivo implantation, in vitro cell seeding and therapeutic applications, wherein said aqueous bioink solution with desired composition is (i) loaded directly on the light-based bioprinting platforms to (ii) create acellular 3D tissues, hydrogel constructs or scaffolds and thereafter (iii) using the 3D tissues, hydrogel constructs or scaffolds for in vivo implantation, in vitro cell seeding or therapeutic applications.
